(19) United States
(12) Patent Application Publication
(10) Pub. No.: US 2019/0275521 A1
(43) Pub. Date: Sep. 12, 2019

(54) APPARATUS FOR OUTER WALL FOCUSING FOR HIGH VOLUME FRACTION PARTICLE MICROFILTRATION AND METHOD FOR MANUFACTURE THEREOF

(71) Applicant: AGENCY FOR SCIENCE, TECHNOLOGY AND RESEARCH, Singapore (SG)

(72) Inventors: Shireen GOH, Singapore (SG); Shan Mei TAN, Singapore (SG)

(21) Appl. No.: 16/319,276

(22) PCT Filed: Jul. 21, 2017
(86) PCT No.: PCT/SG2017/050373

§ 371 Date: Jan. 18, 2019

(30) Foreign Application Priority Data
Jul. 21, 2016 (SG) 10201606028T

(51) Int. Cl. B01L 3/00; B01D 21/26; B04B 5/10; C12M 3/06; C12M 1/00; C12M 1/26

(52) U.S. Cl. CPC B01L 3/502753; B01L 2300/0877; B04B 5/10; B01L 3/50273; C12M 23/16; C12M 29/10; C12M 33/14; B01L 3/502707; B01L 2200/027; B01L 2200/0652; B01L 2200/12; B01L 2300/0681; B01L 2400/0403; B01L 2300/0864; B01D 21/26

(57) ABSTRACT

An apparatus for microfiltration and a scalable method for manufacture of an inertial microfluidic device for such microfiltration apparatus are provided. The apparatus for microfiltration includes one or more inertial microfluidic devices, each including a plurality of spirals of a microfluidic channel. At least one of the inertial microfluidic devices is configured to utilize outer wall focusing for high volume fraction microfiltration of particles. In an embodiment, multiple inertial microfluidic devices are connected in sequence for combined inner wall and outer wall focusing. The scalable method for manufacture of the inertial microfluidic device includes micromachining on a polycarbonate-based substrate a rectangular spiral microchannel having one or more input channels and a plurality of output channels configured to utilize high volume fraction outer wall focusing for microfiltration of particles.

### PRIORITY CLAIM

This application claims priority from Singapore Patent Application No. 10201606028T filed on 21 Jul. 2016.

### TECHNICAL FIELD

The present invention generally relates to microfiltration systems, and more particularly relates to method and apparatus for outer wall focusing at high particle volume fractions to enable high performance particle microfiltration at low shear stress.

### BACKGROUND OF THE DISCLOSURE

Inertial microfluidics has recently gained interest in the microfluidic community because inertial microfluidics generally occurs in channels with characteristic length scales of the order of ˜100 μm with a throughput of approximately 1 ml min⁻¹ making it technologically feasible for macroscopic applications. Therefore, inertial microfluidics based microfiltration for high particle volume fractions has become important for biotechnology and blood applications.

Most inertial microfluidics applications typically involve only particles or cells at dilute concentrations (<0.5 vol %) where the particles are considered to be non-interacting as inertial focusing is integral to inertial microfluidics. Inertial focusing is difficult to achieve at high particle volume fractions because particle-particle interactions defocus the particles.

A trapezoidal spiral channel microfiltration device with skewed Dean's profile has been shown to filter Chinese Hamster Ovary (CHO) cells to the outer wall of the spiral channels with 75% efficiency at cell concentrations of 10⁸ cells/mL. However, such efficiency is not sufficient for many applications and trapezoidal spiral channels are difficult to manufacture and, thus, non-scalable.

Thus, what is needed is a scalable inertial microfluidics device for high particle volume fraction to achieve high throughput microfiltration. Furthermore, other desirable features and characteristics will become apparent from the subsequent detailed description and the appended claims, taken in conjunction with the accompanying drawings and this background of the disclosure.

### SUMMARY

In accordance with the present invention, an apparatus for microfiltration is provided. The apparatus for microfiltration includes one or more inertial microfluidic devices each device including a plurality of spirals of a rectangular microfluidic channel. At least one of the inertial microfluidic devices is configured to utilize outer wall focusing for microfiltration of particles.

In accordance with another aspect of the present invention, a method for manufacture of an inertial microfluidic device is provided. The method includes micromachining on a rigid material substrate a rectangular spiral microchannel having one or more input channels and a plurality of output channels configured to utilize outer wall focusing for microfiltration of particles.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 depicts a planar view of an illustration of a small-scale perfusion filter including a conventional inertial microfluidic filter.

FIG. 2 depicts a top planar view of an illustration of a conventional membrane-less inertial microfluidic filter.

FIG. 3 depicts a top planar view of an illustration of an outer wall focusing inertial microfluidic filter in accordance with a present embodiment.

FIG. 4 depicts a top planar view of the outer wall focusing inertial microfluidic filter illustrated in FIG. 3 in accordance with the present embodiment.

FIG. 5, comprising FIGS. 5A and 5B, depict high volume fraction microfiltration wherein FIG. 5A depicts outer wall focusing in accordance with the present embodiment and FIG. 5B depicts conventional inner wall focusing.

FIG. 6 depicts a graph of particle volume fraction vs. particle distribution within the channel from OW (0%) to IW (100%) for the inertial microfluidic filter in accordance with the present embodiment.

FIG. 7 depicts a top planar view of an illustration of a prior art spiral trapezoidal channel device.

FIG. 8 a bar graph of the separation efficiency of the prior art device illustrated in FIG. 7 at various cell volume fractions.

FIG. 9 a bar graph of the separation efficiency at various cell volume fractions of the device of FIG. 3 in accordance with the present embodiment.

FIG. 10 a bar graph of the filter efficiency of the prior art device illustrated in FIG. 7 as compared to the device of FIG. 3 in accordance with the present embodiment.

FIG. 11 depicts graphs of comparable growth, viability and productivity curves for an unfiltered CHO DG44 cell line producing Herceptin and a CHO DG44 cell line producing Herceptin filtered in accordance with the present embodiment.

FIG. 12 depicts a top planar illustration of combined outer wall focusing and inner wall focusing inertial microfluidic devices in accordance with the present embodiment.

FIG. 13 depicts a front left top perspective view of a six well plate implementation of the inertial microfluidic devices of FIG. 12 in accordance with the present embodiment.

FIG. 14 depicts an illustration of a continuous apheresis device utilizing one or more inertial microfluidic devices in accordance with the present embodiment.

FIG. 15 depicts an illustration of a small volume blood centrifuge utilizing one or more inertial microfluidic devices in accordance with the present embodiment

And FIG. 16 depicts an illustration of a perfusion microbioreactor utilizing inertial microfluidic devices in accordance with the present embodiment.

Skilled artisans will appreciate that elements in the figures are illustrated for simplicity and clarity and have not necessarily been depicted to scale.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of the present embodiment to present applications of outer wall focusing in inertial microfluidics occurring at high particle volume fractions in rectangular spiral channels of microfluidic devices for improving cell microfiltration performance. High particle volume fraction refers to particle volume fractions greater than 10⁷ particles per milliliter (cells/mL) and present cell microfiltration applications have resulted in a greatly improved filter efficiency. For example, using green fluorescent protein (GFP) producing Chinese Hamster Ovary (CHO) cells at high volume fraction of 10⁸ cells/mL, a filter efficiency of greater than 98% has been achieved while prior experiments with GFP producing CHO cells at 10⁸ cells/mL have been unable to achieve 75% filter efficiency.

Studies on inertial focusing at cell volume fractions above 10⁷ cells/mL are difficult to perform because fluorescent microspheres tend to aggregate at high concentrations. Chinese Hamster Ovary (CHO) cells with green fluorescent proteins (GFP) have been used to circumvent this limitation and to also serve as a more accurate mechanical model for soft biological cells.

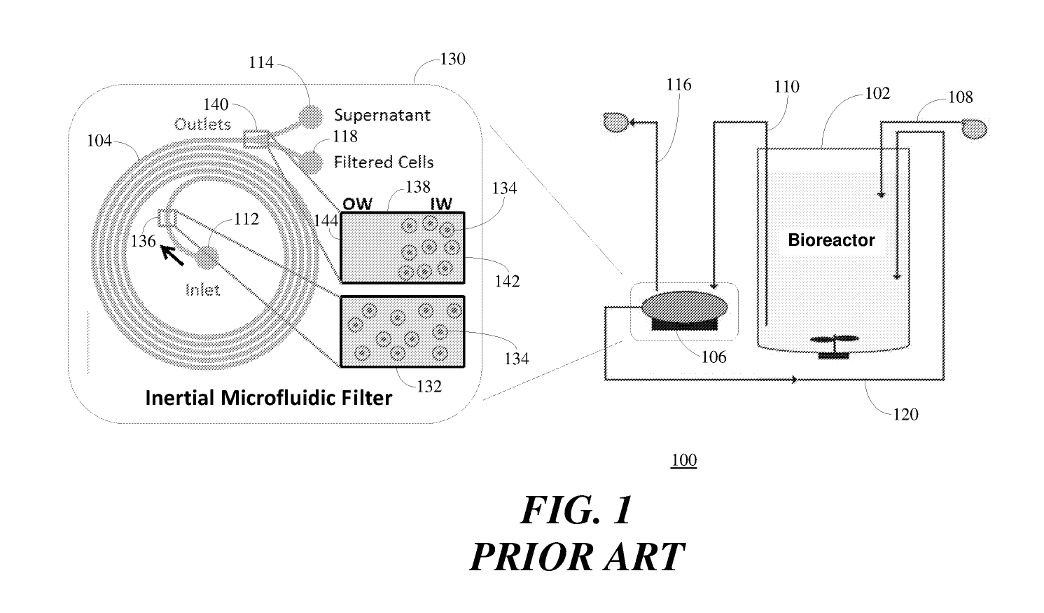

Referring to FIG. 1, a planar view 100 of an illustration of a small-scale perfusion filter is depicted. The small-scale perfusion filter includes a bioreactor 102 and a conventional inertial microfluidic filter 104 serving as a centrifuge 106. The bioreactor 102 is connected to an input 108 for receiving an input of media by perfusion. The bioreactor 102 is also connected to an output 110 for providing a perfused output of cells to the microfluidic filter 104.

The output 110 of the bioreactor 102 provides the perfused output of cells to an inlet 112 of the microfluidic filter 104 as shown in the insert illustration 130. The microfluidic filter 104, as shown in the insert illustration 130, is a microfluidic channel formed into a spiral. A supernatant outlet 114 of the microfluidic filter 104 provides a filtered output 116 of harvested media without cells. A filtered cell outlet 118 of the microfluidic filter 104 provides a feed back of cells to a cell concentrate return 120 for return to the bioreactor 102.

An insert illustration 132 shows a top planar view of cells 134 diffused throughout a cross-section 136 of the microfluidic spiral channel of the microfluidic filter 104 near the inlet 112. Another insert illustration 138 depicts a top planar view of a cross-section 140 of the microfluidic spiral channel of the microfluidic filter 104 near the outlets 114, 118 with an inner wall (IW) 142 and an outer wall (OW) 144 of the microfluidic spiral channel. It can be seen in the insert illustration 138 that near the outlets 114, 118 the cells 134 are focused along the inner wall 142 of the microfluidic spiral channel. Most of the cells 134 focused along the inner wall 142 will follow the inner wall 142 and output the microfluidic filter 104 through the filtered cell outlet 114 while contaminants a small portion of the cells 134 will follow the outer wall 144 and output the microfluidic filter 104 through the supernatant cell outlet 118 for return to the bioreactor 102 via the cell concentrate return 120.

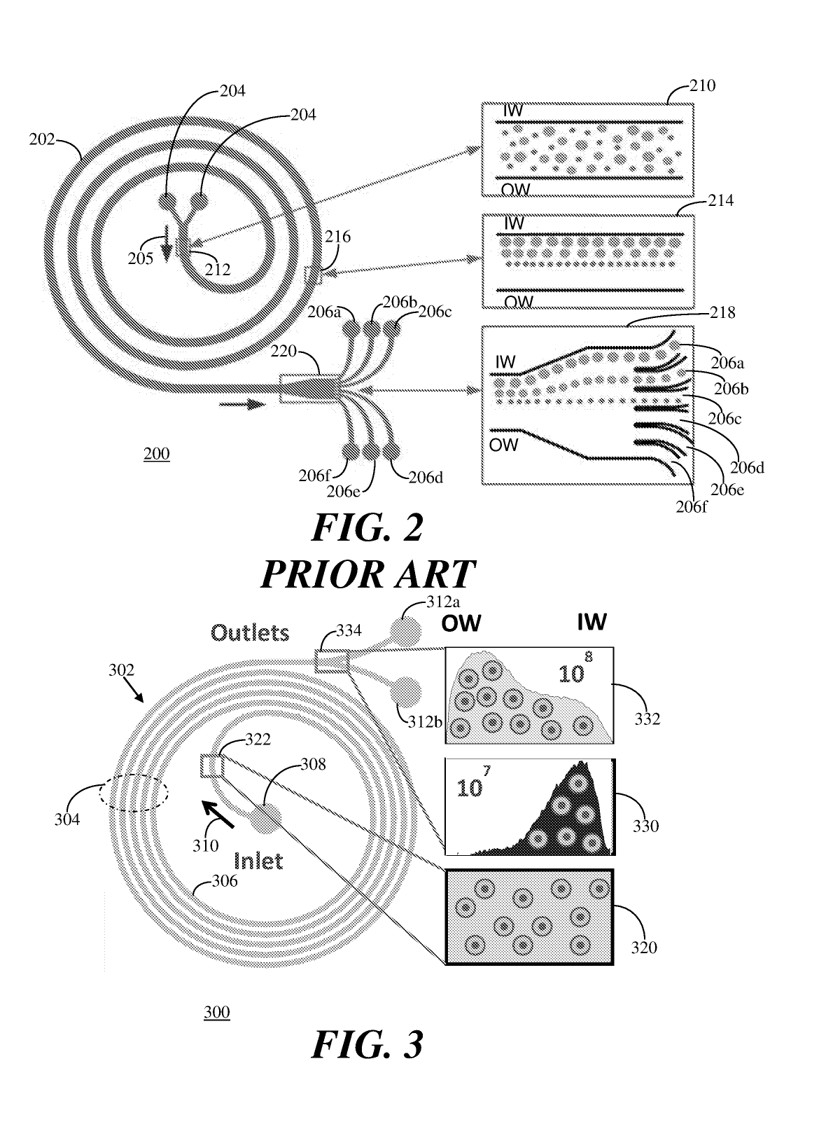

FIG. 2 depicts a top planar view 200 of an illustration of a conventional membrane-less inertial microfluidic filter. The membrane-less inertial microfluidic filter consists of a spiral microfluidic channel 202 for flowing particles from one or more inlets 204 in a direction 205 to one or more outlets 206 (identified as outlets 206a to 2060. A first insert illustration 210 shows a top planar view of particles in a cross-section 212 of the microfluidic spiral channel 202 near the inlets 204. While the particles in the cross-section 212 include particles of different sizes, the particles are evenly diffused throughout the cross-section 212.

A second insert illustration 214 shows a top planar view of particles in a cross-section 216 of the microfluidic spiral channel 202 approximately two-thirds of the distance from the inlets 204 to the outlets 206. The particles in the cross-section 216 have become aligned in the microfluidic spiral channel 202 by size where the larger particles are aligned along an inner wall (IW) and the smallest particles depicted are aligned about mid-channel. A third insert illustration 218 shows a top planar view of particles in a cross-section 220 of the microfluidic spiral channel 202 which includes the outlets 206a to 206f. As the outlets 206 fan out, the larger particles exit through the outlet 206a which includes the inner wall (IW), the next larger particles exit through the outlet 206b and the smallest particles shown exit through the outlet 206c.

Referring to FIG. 3, a top planar view 300 depicts an illustration of an outer wall focusing inertial microfluidic filter 302 in accordance with a present embodiment. The inertial microfluidic filter 302 consists of a plurality of spirals 304 of a microfluidic channel 306 for flowing liquid, fluid or media having particles or cells from an inlet 308 in a direction 310 to two outlets 312 (identified as outlets 312a to 3120. A first insert illustration 320 shows a top planar view of cells as particles in media in a cross-section 322 of the spiral rectangular microfluidic channel 306 near the inlet 308. While the cells in the cross-section 322 include cells of different sizes, the cells are evenly diffused throughout the cross-section 322 as shown in the insert illustration 320. Also, while the microfluidic channel 306 is rectangular in shape, spirals of trapezoidal shaped microfluidic channels where the height of the channel is constant and one or both walls slope inwardly or outwardly from a top surface of the channel to a bottom surface of the channel may also be utilized in accordance with the present embodiment.

A second insert illustration 330 and a third insert illustration 332 show top planar views of cells as particles in a cross-section 334 of the spiral rectangular microfluidic channel 306 near the outlets 312a and 312b. The second insert illustration 330 depicts inertial focusing of cells when approximately 10⁷ cells/mL are flowing through the microfluidic channel which translates to a volume fraction of cells in the spiral rectangular microfluidic channel 306 of approximately 1.7% volume fraction. It can be seen that when the volume fraction of cells in the spiral rectangular microfluidic channel 306 is approximately 1.7%, the inertial focusing of cells is substantially inner wall (IW) focusing.

The third insert illustration 332 depicts cell alignment when approximately 10⁸ cells/mL are flowing through the microfluidic channel and the volume fraction of cells in the spiral rectangular microfluidic channel 306 is approximately 17% volume fraction. Thus, it can be seen that when the volume fraction of cells in the spiral rectangular microfluidic channel 306 of the inertial microfluidic filter 302 in accordance with the present embodiment is approximately 17% volume fraction, the inertial focusing of cells is no longer inner wall (IW) focusing but advantageously shifts to outer wall (OW) focusing. While we have been discussing microfiltration of media having cells, the microfiltration device 302 could be used for microfiltration of any liquid having particles of any kind, such as fluid with particles (e.g., microfiltration of dust particles in water) or media with cells. Also, without limiting applications of the microfiltration device, the preferable ratio of particle diameter to height of the microchannel (i.e., hydrodynamic diameter) is approximately 0.01 to 0.5. Also, while we have been discussing a microfiltration device with one inlet and two outlets, any number of inlets and outlets could be provided and the number of outlets could be greater than, equal to or less than the number of inlets. Also, while FIG. 3 depicts 1.7% volume fraction and 17% volume fraction, the shift to outer wall focusing in accordance with the present embodiment can occur at volume fractions as low as 5% volume fraction and, depending on the radius of particles and the particle interaction in the media, can occur as low as 1% volume fraction.

Inertial focusing occurs on the inner wall of a rectangular spiral channel due to the balance between Dean's force and shear gradient force. However, when the particle volume fraction is increased to high concentrations (e.g., 10⁸ cells/mL), the equilibrium position of the particle shifts from inner wall focusing as shown in the insert illustration 330 to outer wall focusing as shown in the insert illustration 332. The outer wall focusing at high volume fraction appears to be caused by particle-fluid interactions due to the high volume fraction of particles in the suspension. The close proximity of particles to each other inadvertently modifies the flow profile, leading to a switch from inner wall focusing to outer wall focusing. This switch from inner wall focusing to outer wall focusing occurs in rectangular shaped and trapezoidal shaped microfluidic channels where the height of the channel is constant.

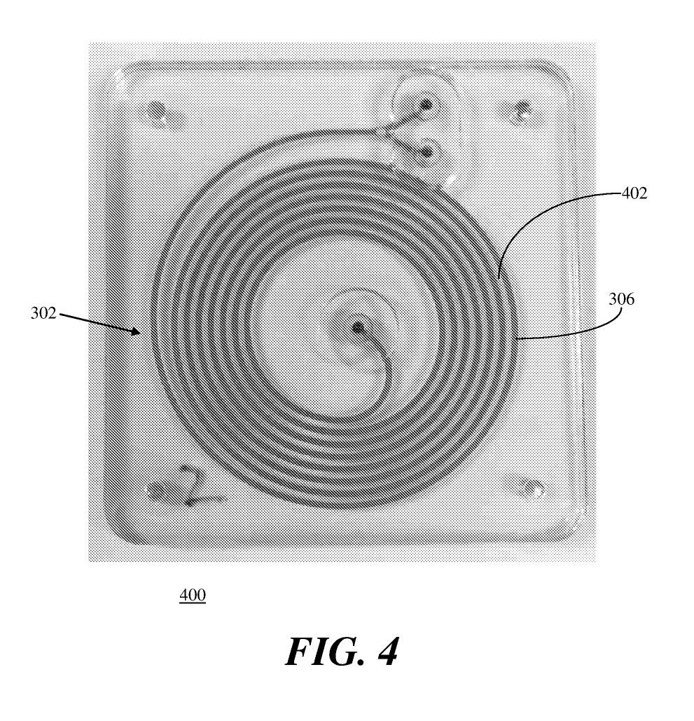

FIG. 4 depicts a top planar view 400 of the outer wall focusing inertial microfluidic filter 302 illustrated in FIG. 3 in accordance with the present embodiment. The rectangular microchannel 306 is micromachined on a polycarbonate substrate using computer numerical controlled (CNC) micromilling. A polycarbonate substrate is selected because polycarbonate is biocompatible, can be mass-prototyped and is less likely to deform during operation as compared to softer PDMS devices. In addition, micromachining a rectangular microchannel in a plurality of spirals on a polycarbonate-based substrate provides a highly scalable method of fabrication. Other rigid material could be used such as thermoplastic materials or other polycarbonate materials to provide similar scalable advantages as the polycarbonate substrate. Also, while rigid materials are preferred for scalable manufacture, one or more non-rigid walls could be provided for the rectangular microchannel 306. However, such flexible material may result in a more diffuse focusing edge and/or a wider focusing width than using rigid materials for all walls of the microchannel 306.

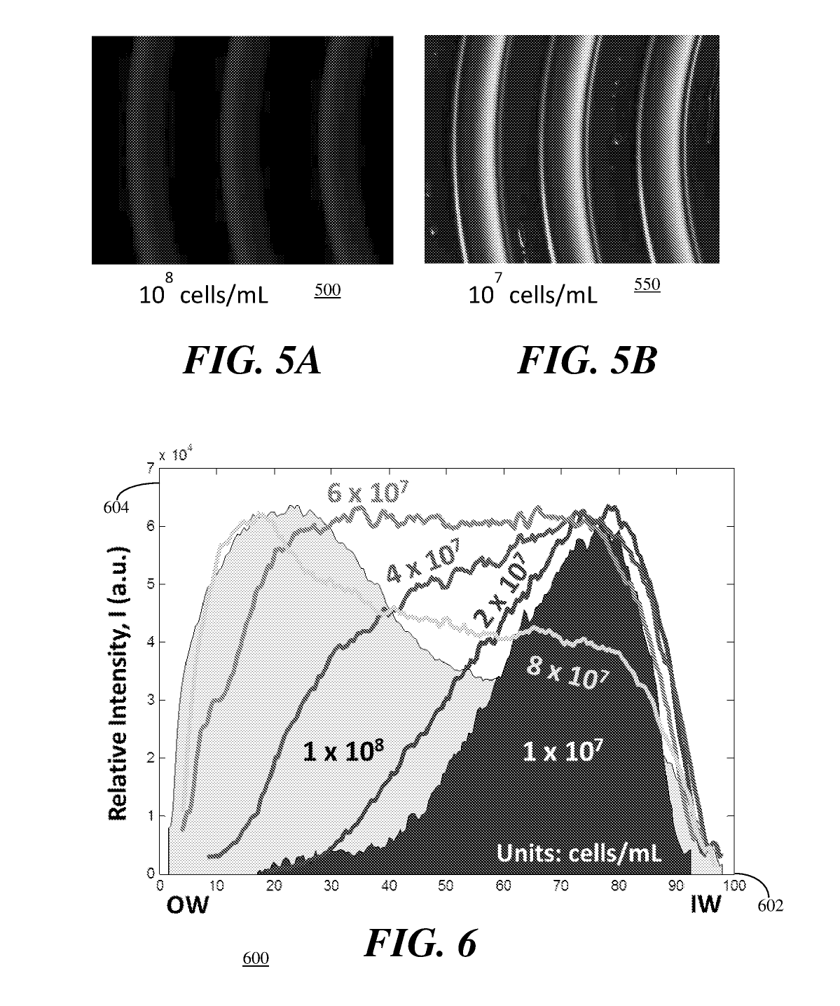

FIG. 5, comprising FIGS. 5A and 5B, fluorescent optical microscope images 500, 550 at four times magnification captured by a monochrome camera are depicted. The image 500 depicts a flow of CHO cells with GFP in rectangular spiral microchannels of a polycarbonate microfilter in accordance with the present embodiment where the high cell volume fraction is approximately 17% (i.e. a concentration of CHO cells of 10⁸ cells/mL). The image 500 depicts a flow of CHO cells with GFP in rectangular spiral microchannels of a polycarbonate microfilter where the cell volume fraction is approximately 1.7% (i.e., a concentration of CHO cells of 10⁷ cells/mL). To determine the cell volume fraction, the images 500, 550 were analyzed using a proprietary graphical user interface (GUI) written in MATLAB. Cell counting was performed using a ViCell™ automated cell counter manufactured by Beckman Coulter, Inc. of Indiana, USA.

FIG. 6 depicts a graph 600 of fluorescence signal versus relative position along the microchannel 306 within the inertial microfluidic filter 302. The position along a floor of the rectangular microchannel 306 is plotted along an x-axis 602 from “0” which indicates the outer wall (OW) to 100 which indicates the inner wall (IW). The fluorescence signal is plotted along a y-axis 604 as relative intensity of the fluorescence. As can be seen, as the cell volume fraction is increased from a CHO cell concentration of 1×10⁷ cells/mL to 1×10⁸ cells/mL in 2×10⁷ cells/mL steps, the position of the cells shifts from inward focusing along the inner wall to outward focusing along the outer wall.

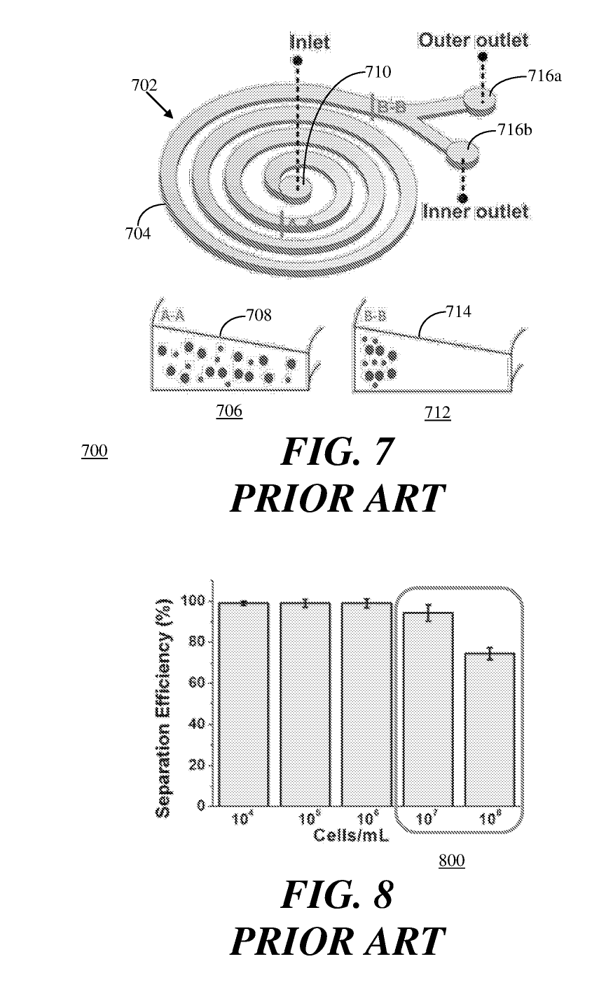

Outer wall focusing has been observed in trapezoidal spiral channels at similar flow rates but at low cell volume fractions. Referring to FIG. 7, a planar view 700 depicts a top planar view 700 of an illustration of one such prior art spiral trapezoidal channel device 702. Cross-sections of the trapezoidal channel 704 are shown in the insert illustration 706 (an illustration of a cross-section 708 near the inlet 710) and the insert illustration 712 (an illustration of a cross-section 714 near the outlets 716a, 716b). It appears that the outer wall focusing in the spiral trapezoidal channel device 702 is caused by a skewed Dean's secondary flow profile in a trapezoidal channel. As can be seen from a bar graph 800 in FIG. 8 of the separation efficiency of the spiral trapezoidal channel device 702, the separation efficiency is consistently high at low CHO cell concentrations up to 10⁶ cells/mL but decreases as the cell concentration increases. For example, at a cell concentration of 10⁸ cells/mL, the separation efficiency has dropped to 74.8%.

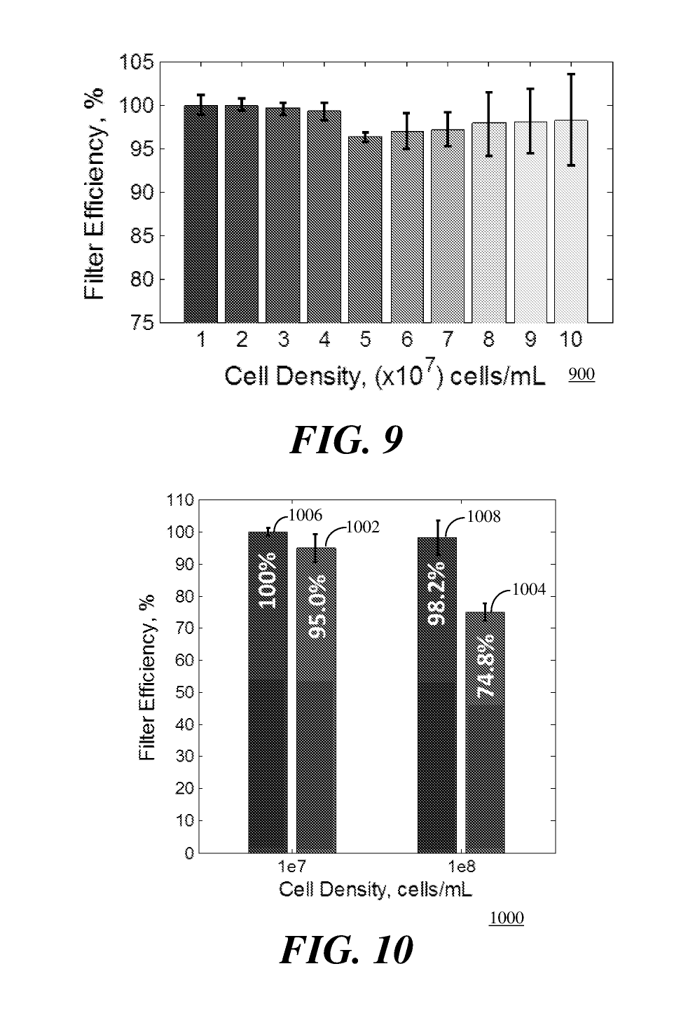

The spiral trapezoidal channel device 702 is unable to filter CHO cells efficiently at 10⁸ cells/mL (only ˜75% separation efficiency). By utilizing outer wall focusing and optimized channel dimensions, the inertial microfluidic filter 302 can achieve 98.2% filter efficiency at CHO cell concentrations of 10⁸ cells/mL and a filter efficiency >95% for all cell concentrations, even for cell concentrations within the transition from inner wall focusing to outer wall focusing as shown in FIG. 9. Referring to FIG. 9, a bar graph 900 of the separation efficiency at various CHO cell concentrations between 10⁷ cells/mL and 10⁸ cells/mL of the outer wall focusing inertial microfluidic filter 302 in accordance with the present embodiment is depicted. Unlike the spiral trapezoidal channel device 702 where the outer wall focusing is caused by a skewed Dean's secondary flow profile in the trapezoidal channel, the outer wall focusing of the inertial microfluidic filter 302 which appears to be caused by particle-fluid interactions causing a distortion of the Dean's secondary flow profile and by increased particle-particle interactions in the non-dilute regime presents a fairly consistent high filter efficiency greater than 95%, even in the cell concentrations between 10⁷ cells/mL and 10⁸ cells/mL where the cells transition from inner focusing to outer focusing as shown in the bar graph 900.

Referring to FIG. 10, a bar graph 1000 summarizes the filter efficiency comparison between the spiral trapezoidal channel device 702 (bars 1002, 1004) as compared to the outer wall focusing inertial microfluidic filter 302 (bars 1006, 1008) in accordance with the present embodiment. The bars 1002, 1006 indicate the filter efficiency of the two devices at 10⁷ cells/mL and the bars 1004, 1008 indicate the filter efficiency of the two devices at 10⁸ cells/mL.

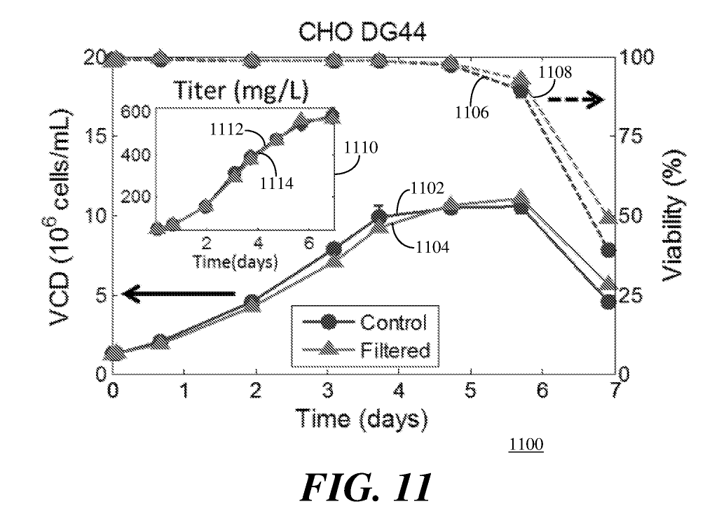

Since outer wall focusing is dominant at lower flow rates (flow rates as low as one-quarter milliliter per minute (i.e., 0.25 mL/min)) in the outer wall focusing inertial microfluidic filter 302, the filtered cells will experience very low shear stress (<0.5 Pa). In addition, cells filtered with the outer wall focusing inertial microfluidic filter 302 are advantageously capable of maintaining the same growth rate and productivity as unfiltered (control) cells. Referring to FIG. 11, an illustration 1100 depicts graphs of comparable growth, viability and productivity curves for an unfiltered CHO DG44 cell line producing Herceptin and a CHO DG44 cell line producing Herceptin filtered in accordance with the present embodiment. A graph 1101 plots growth curves 1102, 1104 and viability curves 1106, 1008 for filtered and unfiltered (control) CHO DG44 cell lines producing Herceptin, respectively. A graph 110 inset in the graph 1101 plots productivity curves 1112, 1114 for the filtered and unfiltered cell lines, respectively, and shows that for both cell lines, the productivity/product titer is unaffected by filtration through the outer wall focusing inertial microfluidic filter 302.

The outer wall focusing inertial microfluidic filter 302 was fabricated using CNC machined microchannels on polycarbonate substrates which has the advantage of being compatible with mass production (i.e., highly scalable) and is less likely to deform during the operation compared to softer PDMS devices.

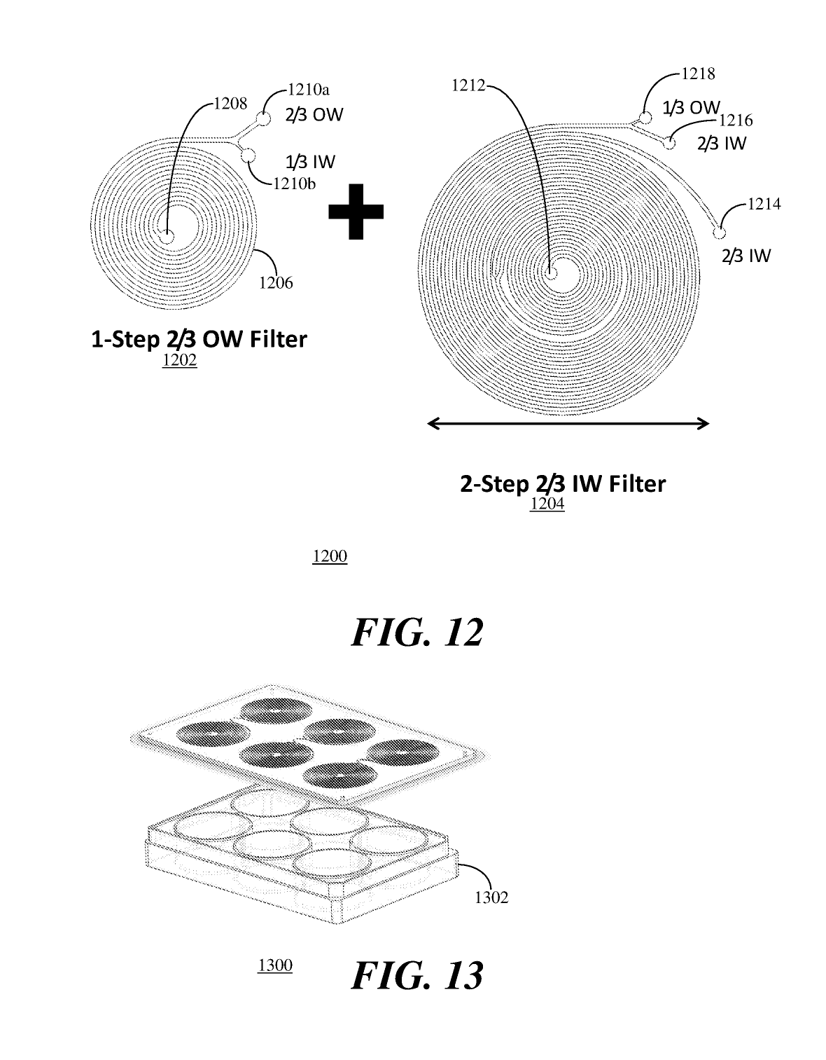

FIG. 12 depicts a top planar illustration 1200 of combined outer wall focusing and inner wall focusing inertial microfluidic devices 1202, 1204 in accordance with the present embodiment. The outer wall focusing inertial microfluidic device 1202 is configured to utilize outer wall focusing for microfiltration of cells from media by having five to seven spirals of a rectangular microchannel 1206 connecting one inlet 1208 to two outlets 1210a, 1210b. The outlet 1210a is an outer wall focused outlet having a width substantially two-thirds of the width of the rectangular microchannel 1206 and the outlet 1210b is an inner wall focused outlet having a width substantially one-third of the width of the rectangular microchannel 1206. While this particular embodiment has the outer wall focused outlet 1210a having a width substantially two-thirds of the width of the rectangular microchannel 1206 and the inner wall focused outlet 1210b having a width substantially one-third of the width of the rectangular microchannel 1206, these widths are exemplary and any widths between one-tenth ( 1/10) of the width of the rectangular microchannel 1206 to one-half (½) of the width of the rectangular microchannel 1206 can be used in accordance with the present embodiment.

The inertial microfluidic device 1204 is a two-step inertial microfluidic device, each step being an inner wall focusing inertial microfluidic devices having five to seven rectangular spiral channels connecting one inlet to two outlets. An inlet 1212 is the inlet of the first step and is connected to the inner wall focused outlet 1210b of the inertial microfluidic device 1202 to provide additional filtering to remove cells from the media. The inner wall outlet of the first step is a first outlet 1214 of the inertial microfluidic device 1204. The outer wall outlet of the first step is connected to the inlet of the second step and the inner wall and outer wall outlets of the second step are a second outlet 1216 and a third outlet 1218, respectively, of the inertial microfluidic device 1204.

The combination of outer wall focusing and inner wall focusing provides an improved filtration device. In addition, such combined devices can fit on a conventional six well plate 1302 as shown in the front left top perspective view 1300 of FIG. 13 to provide additional capacity. For example, filtration device shown in FIG. 12 can be attached to a microbioreactor such as Ambr(TAP) 15 mL or 250 mL bioreactors manufactured by TAP Biosystems, a part of Sartorius Stedim Biotech of Cambridge, UK. When stacked in the six well configuration on the six well plate 1302, the stacked filtration device can be used to filter 500 mL to 5L bioreactors. Thus, filtration devices in accordance with the present embodiment can be used for filtration of bioreactors from 2 mL bioreactors to 5L bioreactors.

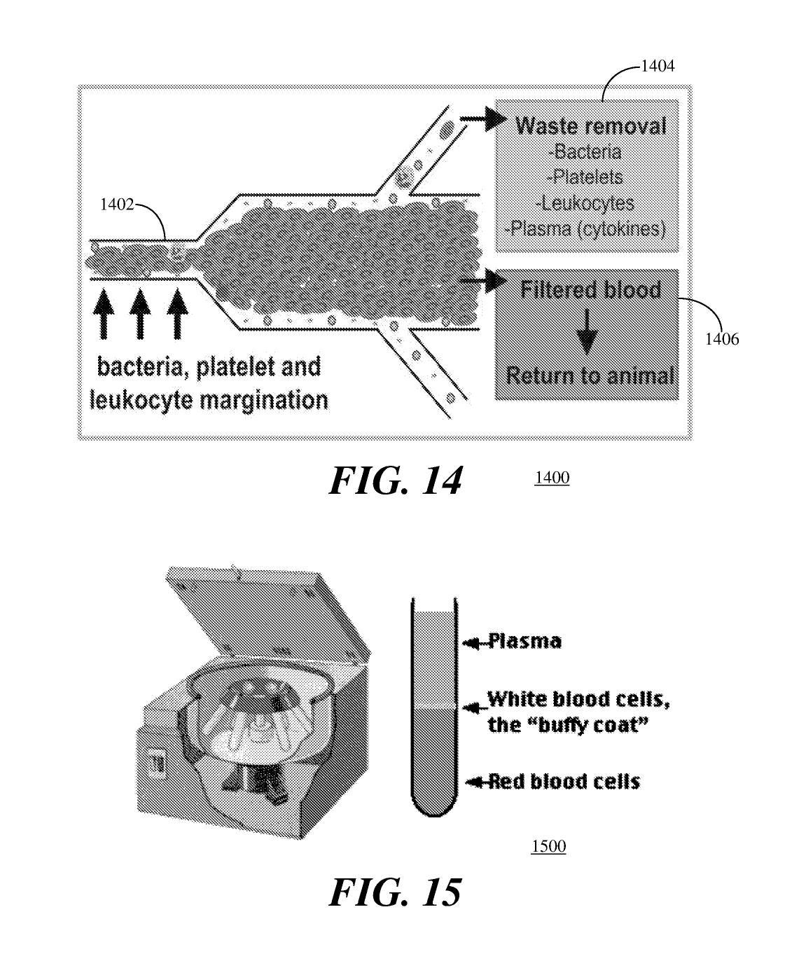

Referring to FIG. 14, an illustration 1400 depicts a continuous apheresis device 1402 utilizing one or more inertial microfluidic devices in accordance with the present embodiment. A blood input 1402 of bacteria, platelet and leukocyte margination received from an animal can be filtered through the one or more inertial microfluidic devices to remove waste particles 1404 from the blood so that the filtered blood 1406 can be returned to the animal. Use of the one or more inertial microfluidic devices in accordance with the present embodiment can increase a conventional microfiltration throughput of 100 μL/minute to 1 μL/minute.

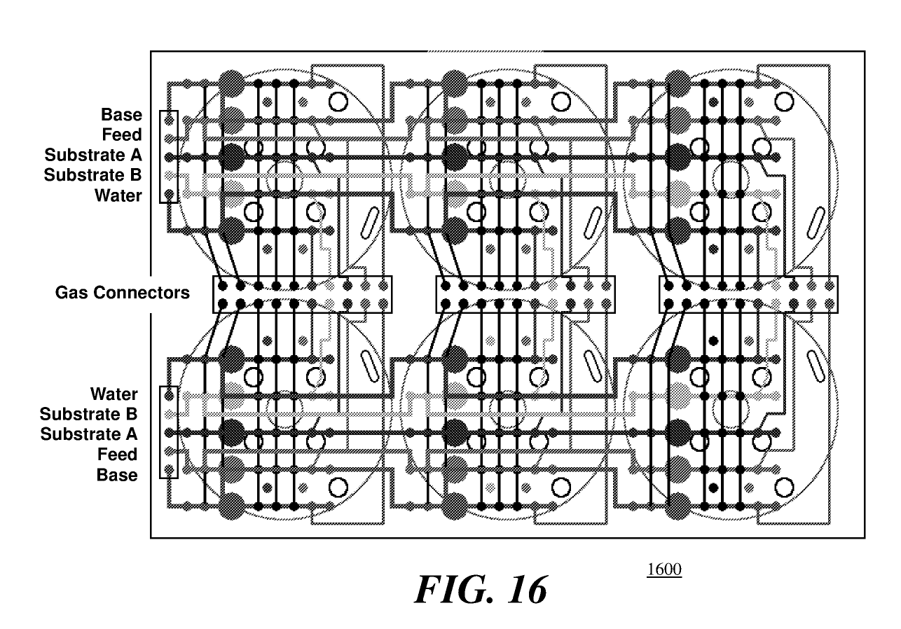

FIG. 15 depicts an illustration 1500 of a small volume blood centrifuge utilizing one or more inertial microfluidic devices in accordance with the present embodiment. The inertial microfluidic devices in accordance with the present embodiment can be utilized for separating of blood constituents at high hematocrit without pre-dilution as shown in the illustration 1500. Use of the one or more inertial microfluidic devices in accordance with the present embodiment can reduce a conventional time for centrifugal small volume blood separation from fifteen minutes with damage to a sample to three minutes with little or no damage to the sample.

As a biotechnology application in biotechnology where high volume fraction cell cultures are prevalent which can advantageously utilize inertial microfluidic devices in accordance with the present embodiment FIG. 16 depicts an illustration 1600 of a perfusion microbioreactor comprising inertial microfluidic devices in accordance with the present embodiment. Use of the one or more inertial microfluidic devices in accordance with the present embodiment can provide a continuous perfusion microbioreactor while conventional perfusion microbioreactors can only provide semi-perfusion.

Thus, it can be seen that the present embodiment provides a highly scalable inertial microfluidics device for high particle volume fraction fluids to achieve high throughput microfiltration. The outer wall focusing in inertial microfluidics in accordance with the present embodiment occurs at high particle volume fractions in rectangular spiral channels of microfluidic devices for improving cell microfiltration performance. High particle volume fraction refers to particle volume fractions greater than 10⁷ particles per milliliter (cells/mL) and cell microfiltration applications utilizing microfiltration devices in accordance with the present embodiment have resulted in a greatly improved filter efficiency.

While exemplary embodiments have been presented in the foregoing detailed description of the invention, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of steps and method of operation described in the exemplary embodiment without departing from the scope of the invention as set forth in the appended claims.

What is claimed is:

1. An apparatus for microfiltration comprising:
one or more inertial microfluidic devices comprising a plurality of spirals of a microfluidic channel wherein at least a first one of the one or more inertial microfluidic devices is configured to utilize outer wall focusing for high volume fraction microfiltration of fluid having particles.

2. The apparatus in accordance with claim 1 wherein the fluid comprises a liquid or media.

3. The apparatus in accordance with claim 1 wherein the high volume fraction microfiltration of fluid comprises the high volume fraction microfiltration at a predetermined flow rate.

4. The apparatus in accordance with claim 3 wherein the predetermined flow rate is greater than one-quarter milliliter per minute.

5. The apparatus in accordance with claim 1 wherein the high volume fraction microfiltration of the media comprises high volume fraction microfiltration of the media at a volume fraction greater than one percent (1%).

6. The apparatus in accordance with claim 5 wherein the high volume fraction microfiltration of the media comprises high volume fraction microfiltration of the media at a volume fraction greater than 1.7%.

7. The apparatus in accordance with claim 6 wherein the high volume fraction microfiltration of the media comprises high volume fraction microfiltration of the media at a volume fraction greater than five percent (5%).

8. The apparatus in accordance with claim 1 wherein the at least a first one of the one or more inertial microfluidic devices comprises a first predetermined number of inlets coupled via the microfluidic channel to a second predetermined number of outlets, wherein at least one of the second predetermined number of outlets is configured to provide an output of an outer wall filtrated portion of the particles.

9. The apparatus in accordance with claim 8 wherein the at least one of the second predetermined number of outlets configured to provide the output of the outer wall filtrated portion of the particles has a width greater than other ones of the second predetermined number of outlets, and wherein widths of each of the second predetermined number of outlets is between one-tenth of a width of the microfluidic channel and one-half of the width of the microfluidic channel.

10. The apparatus in accordance with claim 9 wherein the second predetermined number of outlets comprises two outlets, and wherein a first outlet is an outer wall focused outlet configured to provide the output of the outer wall filtrated portion of the particles and a second outlet is an inner wall focused outlet.

11. The apparatus in accordance with claim 1 wherein at least a second one of the one or more inertial microfluidic devices is configured to utilize inner wall focusing for microfiltration of particles.

12. The apparatus in accordance with claim 11 wherein the at least a second one of the one or more inertial microfluidic devices comprises a first predetermined number of inlets coupled via the plurality of spirals of the microfluidic channel to a second predetermined number of outlets, wherein the second predetermined number is greater than the first predetermined number and at least one of the second predetermined number of outlets has a width greater than other ones of the second predetermined number of outlets to provide an output of an inner wall filtrated portion of the particles.

13. The apparatus in accordance with claim 12 wherein the second predetermined number of outlets comprises two outlets, and wherein a first outlet is an inner wall focused outlet and the second outlet is outer wall focused outlet, and wherein the inner wall focused outlet is the at least one of the second predetermined number of outlets which has a width greater than the outer wall focused outlet.

14. The apparatus in accordance with claim 13 where a width of the inner wall focused outlet is substantially two-thirds of the width of the microfluidic channel and a width of the outer wall focused outlet is substantially one-third of the width of the microfluidic channel.

15. The apparatus in accordance with claim 14 wherein the one or more inertial microfluidic devices comprise one or more groups of three inertial microfluidic devices and wherein a first one of each group of three inertial microfluidic devices comprises an inertial microfluidic device configured to utilize outer wall focusing for microfiltration of particles by having a first predetermined number of spirals of a microfluidic channel connecting one inlet to two outlets, a first outlet being an outer wall focused outlet having a width substantially two-thirds of the width of the microfluidic channel and a second outlet being an inner wall focused outlet having a width substantially one-third of the width of the microfluidic channel, and
wherein a second one and a third one of each group of three inertial microfluidic devices each comprise an inertial microfluidic device configured to utilize inner wall focusing for microfiltration of particles by having a respective second and third predetermined number of spirals of a microfluidic channel connecting one inlet to two outlets, wherein the two outlets comprise an inner wall focused outlet and an outer wall focused outlet and wherein the inner wall focused outlet has a width substantially two-thirds of the width of the rectangular microfluidic channel and the outer wall focused outlet has a width substantially one-third of the width of the microfluidic channel, and
wherein the inlet of the first one of each group of three inertial microfluidic devices is configured to receive an input of unfiltered media having particles, and
wherein the inlet of the second one of each group of three inertial microfluidic devices is configured to receive an input of filtered media from the inner wall focused outlet of the first one of each group of three inertial microfluidic devices, and
wherein the inlet of the third one of each group of three inertial microfluidic devices is configured to receive an input of filtered media from the inner wall focused outlet of the second one of each group of three inertial microfluidic devices, and
wherein the filtered media output from the inner wall focused outlet of the third one of each group of three inertial microfluidic devices is provided as an output from the group of three inertial microfluidic devices.

16. The apparatus in accordance with claim 15 wherein each of the first predetermined number of spirals, the second predetermined number of spirals and the third predetermined number of spirals are selected from the group comprising five spirals, six spirals and seven spirals.

17. The apparatus in accordance with any of the preceding claims wherein the apparatus for microfiltration comprises a continuous apheresis device using microfluidics for separating blood constituents at high hematocrit without pre-dilution.

18. The apparatus in accordance with any of the preceding claims wherein the apparatus for microfiltration comprises a small volume blood centrifuge.

19. The apparatus in accordance with any of the preceding claims wherein the apparatus for microfiltration comprises a perfusion microbioreactor for providing continuous perfusion filtration.

20. The apparatus in accordance with any of the preceding claims wherein the apparatus for microfiltration comprises a small-scale perfusion filter, the perfusion filter further comprising:
a bioreactor configured to receive an input of media comprising particles for perfusion and coupled to the one or more inertial microfluidic devices for providing a perfused output thereto, and
wherein the one or more inertial microfluidic devices filter the perfused output of the bioreactor to provide a harvested output of the media.

21. The apparatus in accordance with claim 20 wherein the one or more inertial microfluidic devices are further coupled to the bioreactor to feed back a cell concentrate to the bioreactor.

22. The apparatus in accordance with any of the preceding claims wherein the microfluidic channel of each of the one or more inertial microfluidic devices comprises a rectangular spiral microchannel having one or more input channels and a plurality of output channels micromachined on a rigid material.

23. The apparatus in accordance with claim 22 wherein the rigid material comprises a material selected from a polycarbonate-based substrate, a material comprising polycarbonate, or a thermoplastic material.

24. A method for manufacture of an inertial microfluidic device comprising micromachining on a rigid substrate a rectangular spiral microchannel having one or more input channels and a plurality of output channels configured to utilize outer wall focusing for high volume fraction microfiltration of particles.

25. The method in accordance with claim 24 wherein the plurality of output channels include at least an outer wall focused output channel, and wherein the inertial microfluidic device is configured to utilize outer wall focusing for high volume fraction microfiltration of particles by micromachining the outer wall focused output channel to have a width greater than widths of all other ones of the plurality of output channels.
